(19) United States
(12) Patent Application Publication
(10) Pub. No.: US 2015/0068941 A1
(43) Pub. Date: Mar. 12, 2015

(54) Containment Clip for Medical Devices

(71) Applicant: AngioDynamics, Inc., Latham, NY (US)

(72) Inventor: Lizabeth K. Caron, Brighton, MA (US)

(73) Assignee: AngioDynamics, Inc., Latham, NY (US)

(21) Appl. No.: 14/022,615

(22) Filed: Sep. 10, 2013

(51) Int. Cl. A61M 25/00

(52) U.S. Cl. CPC A61M 25/002
USPC 206/364

(57) ABSTRACT

A containment clip is provided having securement members for connection to medical device accessories such as guide wire hoops and access accessories such as needles and introducers. The containment clip can be utilized as part of a kit, secured by a hinged connection to one or more medical device accessories.

The present invention relates to an improved containment clip for medical device accessories. More particularly, the present invention relates to a containment clip for securing medical device accessories used for vascular and non-vascular access to target treatment sites in a human body.

### BACKGROUND OF THE INVENTION

Before physicians perform certain medical procedures on a patient, access to a target treatment site must be established. Access techniques are typically designed to advance medical devices to the target treatment site while minimizing the amount of trauma caused to the patient. Access procedures are routine in clinical settings, and access is gained using techniques that utilize a variety of medical device accessories. Those accessories may include an introducer needle, a starter wire, one or more introducers (including valved, non-valved, and peelable introducers), and a guide wire. Target treatment sites may include the vascular system, such as the junction of the right atrium and the superior vena cava, or non-vascular sites, such as the bladder, kidney, biliary duct or an abscess.

An example of a typical procedure for establishing vascular access may be as follows: First, a 21G introducer needle is advanced into a vein. Access to the vein can be verified by the presence of blood, commonly referred to as “flashback” in a proximal chamber of the needle. Once the vein is accessed, a 0.018 inch guide wire can be inserted into the vein through a lumen of the needle. The needle is then back loaded out of the vein and off the 0.018 inch guide wire. With access to the vein established via the guide wire, a 4F introducer can be inserted over the guide wire and into the vein. The introducer consists of a sheath coaxially loaded over a dilator as an assembly for smooth transition into the vein. The dilator and guide wire are then withdrawn back out of the vein and the sheath. The sheath remains in place to provide access for other medical devices. As the medical practitioner completes use of each accessory, they are disposed of in a sharps container.

There are also kits for non-vascular access, such as those required for a drainage catheter. Differences may include needles and introducers that are longer, and the addition of an extra wire commonly referred to as a starter wire. An example of a common procedure for establishing non-vascular access may be as follows: A 21G introducer needle is advanced to the treatment area, such as the bladder, kidney, biliary duct or an abscess. Next, a 0.018 inch guide wire is inserted through the needle. Once the 0.018 inch guide wire is in place, the needle is back loaded off the guide wire and disposed of in a sharps container. Next, a 6F introducer is inserted over the 0.018 inch guide wire to the treatment site. However, because the introducer is longer, a stylette may be required as part of the introducer assembly to function as a stiffening element. Once the introducer is in place, the dilator, stylette, and 0.018 inch guide wire are removed and disposed of, with the sheath remaining in place. Next, a 0.035 inch guide wire is inserted through the sheath and into the treatment area, providing access for a drainage catheter.

Access kits may be sold as a standalone kit or packaged with the primary medical device being used for treatment. The components of the access kit may depend on a number of factors, including the procedure being performed, the anatomy of the patient, the geometry of the primary medical device being used to treat the patient, and the preferences of the medical practitioner performing the access procedure. Medical device kit manufacturers may also offer practitioners the opportunity to order access kits that are customizable for a specific practice.

Generally, each component is individually protected within the kit, typically with a tubular plastic cover. Needles and introducers are commonly enclosed in plastic protectors that extend up to a hub of the device, while the guide wire is commonly contained in a tubular plastic protector commonly referred to as a guide wire hoop. The protectors and the hoops serve multiple functions, including protecting the components during shipment and storage, preventing people from haphazardly sticking or injuring themselves on sharp edges, and providing a surface and structure for the component to be secured into a containment clip.

Containment clips have been used by medical device manufacturers to keep access components secured and bundled together. During shipping and storage, containment is helpful so that components do not move around in the packaging and otherwise become damaged or compromised. Further, in a clinical setting, where a variety of medical devices are laid out in preparation for the procedure, containment clips help the practitioner by keeping accessories for a particular step in a procedure within close proximity to one another, so that they can be lie together in the sterile field and components do not otherwise roll around or become misplaced.

FIG. 11 shows a prior art configuration for a containment clip 201 which secures a guide wire protector 215 containing a guide wire (not shown), a needle protector 212 containing a needle 211, and an introducer protector 210 containing an introducer 209. Medical device manufacturers who assemble and sell access kits will often first order specific kit accessories such as the guide wires, needles and introducers from outside vendors. Once the kit manufacturer receives the specific components, they can be assembled and packaged as a kit for sale. The guide wire is typically provided to the kit manufacturer by the guide wire vendor packaged in a protector and assembled as a guide wire hoop, similar to the guide wire hoops shown in FIGS. 3, 4, 8 and 9. The hoop allows the guide wire to be packaged in a smaller footprint and the gradual curvature of the hoop allows for storage and shipment without kinking the wire. As a result, and as shown in the example of prior art FIG. 11, prior art containment clip 201 designs may require that the guide wire protector 215 be unraveled from hoop form and reconfigured to a horseshoe shape to fit the containment clip 201. This adds additional time, labor, expenses and inefficiencies to the packaging of the kits since the hoop will have to be unraveled and the guide wire repackaged and reshaped to accommodate the containment clip. Further, the horseshoe shaped guide wire packaging 215 causes the guide wire to rest at a more extreme angle of curvature compared to the standard hoop, which can deform the guide wire, compromising its performance and trackability during a procedure. Extreme angles of curvature may also cause cracking of any coatings that may be present on the guide wire. Additionally, for practitioners, handling an oblong containment assembly can be awkward compared to the uniform circular hoop shaped assembly. Even further, withdrawing accessories from prior art clips can be difficult as accessories are crowded and clearance between accessories is minimized.

Therefore, there is a need for an improved containment clip which provides easy handling and access to medical device accessories, while maintaining efficiencies during the kit assembly process.

### SUMMARY OF THE INVENTION

The invention is directed to a containment clip for medical device accessories, and to kits utilizing the same.

In one embodiment, a kit for securing medical device accessories includes a guide wire hoop, a containment clip, and a first medical device accessory. The containment clip has a first and second securement member, the first securement member configured for a hinged connection to the guide wire hoop, and the second securement member configured for an interference fit with the first medical device accessory. The guide wire hoop is secured to the first securement member and the first medical device accessory is secured to the second securement member.

In another embodiment, a containment clip for securing medical device accessories includes a first end portion, an intermediate portion, and a second end portion, the intermediate portion separating the first end portion and the second end portion. A first open-faced securement member is disposed on the first end portion, and a second open-faced securement member is disposed on the second end portion. The first securement member is configured to form a hinged interference fit with a guide wire hoop, and the second securement member is configured to form an interference fit with a medical device accessory.

### BRIEF DESCRIPTION OF THE DRAWINGS

FIG. 1 shows a perspective view of a containment clip according to a first embodiment of the invention;

FIG. 2 shows a side view of the containment clip shown in FIG. 1;

FIG. 3 shows a top view of an access kit assembly utilizing the containment clip shown in FIG. 1;

FIG. 4 shows a perspective view of the access kit shown in FIG. 3;

FIGS. 5A-5C shows diagrams of side views of the access kit shown in FIG. 3: FIG. 5A shows the containment clip assuming an angle of approximately 0 degrees with respect to the guide wire hoop, FIG. 5B shows the containment clip assuming an angle of approximately 45 degrees with respect to the guide wire hoop, and FIG. 5C shows the containment clip assuming an angle of approximately 90 degrees with respect to the guide wire hoop;

FIG. 6 shows a perspective view of a containment clip according to a second embodiment of the invention;

FIG. 7 shows a side view of the containment clip shown in FIG. 7;

FIG. 8 shows a top view of an access kit assembly utilizing the containment clip shown in FIG. 7;

FIG. 9 shows a perspective view of the access kit shown in FIG. 8;

FIGS. 10A-10E show diagrams of side views of the access kit shown in FIG. 8: FIG. 10A shows the containment clip assuming an angle of approximately 0 degrees with the guide wire hoop, FIG. 10B shows the containment clip assuming an angle of approximately 30 degrees with respect to the guide wire hoop, FIG. 10C shows the containment clip and the starter wire assuming an angle of approximately 45 degrees with respect to the guide wire hoop, FIG. 10D shows the containment clip assuming an angle of approximately 90 degrees with respect to the guide wire hoop and the starter wire assuming an angle of approximately 45 degrees with respect to the guide wire hoop, and FIG. 10E shows the containment clip and the starter wire assuming an angle of approximately 90 degrees with respect to the guide wire hoop; and

FIG. 11 shows a top view of an access kit assembly utilizing a prior art containment clip.

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein is a containment clip for medical devices.

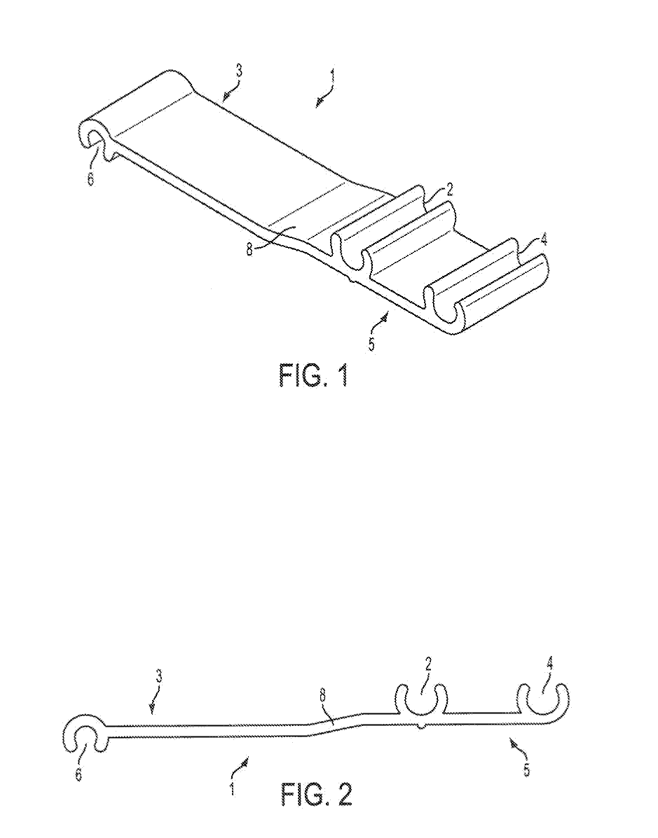

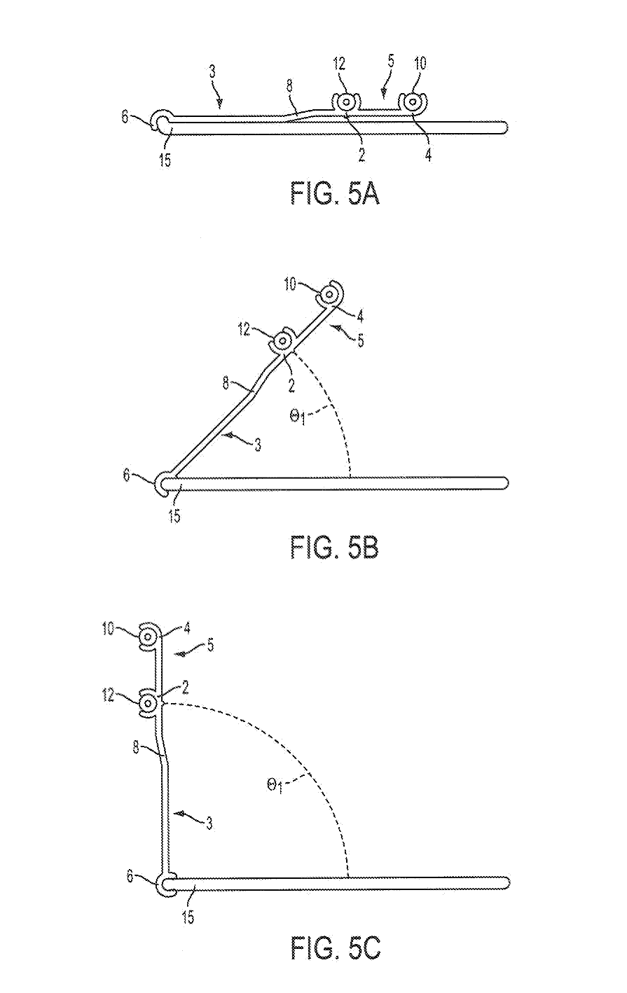

A first embodiment of a containment clip 1 according to the invention is shown in FIG. 1. The containment clip 1 has a first end portion 3, a second end portion 5, and a riser portion 8 separating the first end portion 3 and the second end portion 5. As better illustrated in the side view of FIG. 2, the riser portion 8 elevates the second end portion 5 to a plane slightly higher than the plane of the first end portion 3. The function of the riser portion will be explained in further detail below with reference to FIG. 5A. Still referring to FIGS. 1 and 2, securement members 2, 4 are configured on the containment clip 1 for securing medical device accessories. An additional securement member 6 is configured on the containment clip 1 for securing a hinged connection between the containment clip 1 and a guide wire hoop. The securement clip 1 is preferably made of plastic via an injection molding processes, but can be made of any other suitable materials such as metals.

The securement members 2, 4, 6 can take any shape that will form an interference fit with the corresponding accessory. In a preferred embodiment, the securement members 2, 4, 6 have a geometry to form a snap fit with the corresponding accessory, which is loose enough to accept the accessory using a light pressing motion, but tight enough to secure the accessory in position during shipment and handling by medical professionals. Preferably, as shown in FIGS. 1 and 2, the securement members 2, 4, 6 are open-faced and C-shaped, but they may also be open-faced with a series of flat or contour surfaces, or otherwise have a closed shape geometry capable of achieving an interference or other suitable fit for securing corresponding medical device accessories. The securement members 2, 4, 6 can be configured to any spacing to accommodate medical device accessories according to the kit being packaged. For example, securement members can be resized so that the same clip can fit accessories of different sizes. Securement members may also be breakable, or shaped to fit accessories that have non-circular outer surfaces.

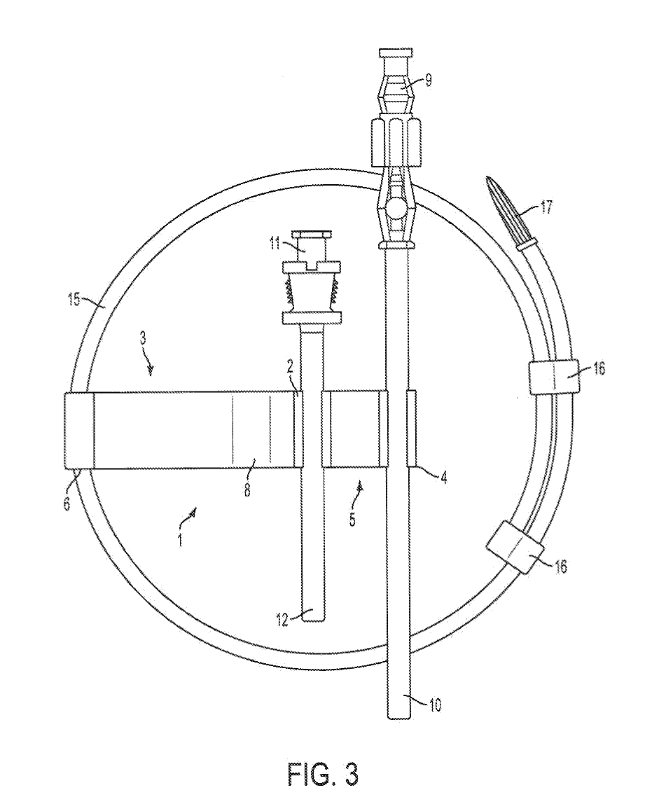

An example of a configuration for a vascular access kit utilizing the containment clip 1 is shown in FIG. 3. The containment clip 1 has a securement member 6 on the first end portion 3 with a geometry forming a snap fit with a guide wire hoop 15. The guide wire hoop 15 is a flexible plastic tubular structure containing a guide wire (not shown), and the hoop retains its circular shape via a pair of snap fit clips 16. The guide wire hoop will typically include a guide wire straightener 17 for protecting the tip of the guide wire. Securement members 2, 4 are also positioned on the second end portion 5 to secure an introducer 9 and an access needle 11. The introducer 9 and access needle 11 are housed in protectors 10, 12, and the protectors snap fit into their respective securement members 2, 4. In a preferred embodiment, the securement member 6 on the first end portion 3 faces a direction opposite of the securement members 2, 4 on the second end portion. This configuration allows for easy attachment of the containment clip 1 to the guide wire hoop 15 using a simple downward pressing motion, as well as easy attachment of the accessories 9, 11 to the containment clip 1 using a downward pressing motion, with the accessories 9, 11 resting on the top side of the hoop.

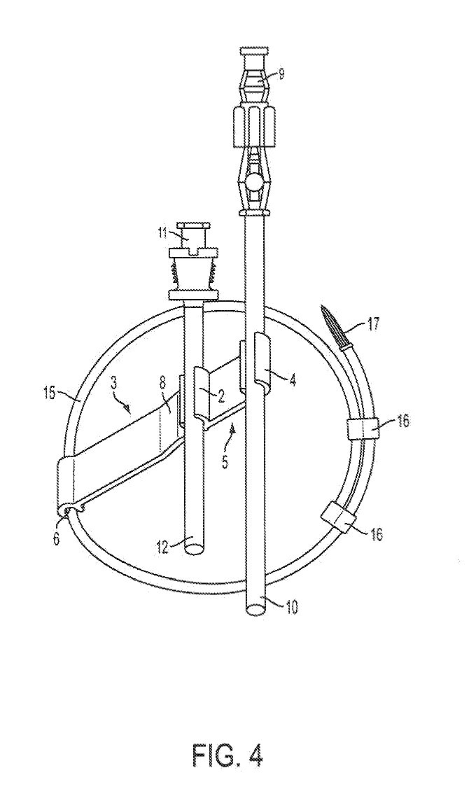

As shown in FIG. 4, the containment clip 1 forms a hinged connection with the guide wire hoop 15, allowing the containment clip 1 to swing up and away from the hoop 15, allowing for easy access to accessories 9, 11. The interference fit between the securement member 6 and the guide wire hoop 15 is configured such that the containment clip 1 can be raised to any elevated position, and the containment clip 1 will remain in that position until further manipulation by the user. The hoop also functions as a base for supporting the containment clip 1. The circular shape of the hoop 15 provides a sturdy base for the containment clip 1 and accessories 9, 11 such that the accessories 9, 11 can rest at any level of elevation relative to the guide wire hoop 15.

As a result of selective elevation of the clip, access to the accessories 9, 11 is improved, as shown the diagrams shown in FIG. 5A-5C illustrate. In FIG. 5A, the clip 1 has not been elevated. Here, the function of the riser 8 is best illustrated, which is to ensure that the protectors 10, 12 and their corresponding accessories rest on top of the hoop 15, providing easy access for medical professionals. As shown in FIGS. 5B and 5C, the containment clip 1 can be elevated to various positions which will assume an angle Θ₁ with the hoop 15. Angles Θ₁ can be approximately 45 degrees as shown in FIG. 5B, 90 degrees as shown in FIG. 5C, or elevations forming angles Θ₁ between 0 and 90 degrees. As desired, angles Θ₁ past 90 degrees can also be formed.

Elevating the accessories provides practitioners with more efficient access since the accessories are now contained in a central location and elevated above sterile barriers and other tools that may be laid out in the immediate vicinity of the access kit. With the accessories in an elevated position, they reside in a higher plane for easy identification and more simplified retrieval from their protectors. The hinged connection also improves separation between accessories, which might otherwise be crowded according to prior art containment clip configurations. A further advantage of the containment clip 1 according to the present invention is that the guide wire hoop 15 does not require reconfiguration by the kit manufacturer. As mentioned above, the guide wire components are typically provided to kit manufacturers in hoop form. As illustrated in FIGS. 1-4, the containment clip 1 can snap onto the guide wire hoop 15 as supplied without the reconfiguration required by prior art clips (see FIG. 11). Therefore, the present invention avoids the additional time, cost and inefficiencies associated with hoop reconfiguration, while further avoiding performance issues that may be associated with a more severe curvature of the guide wire.

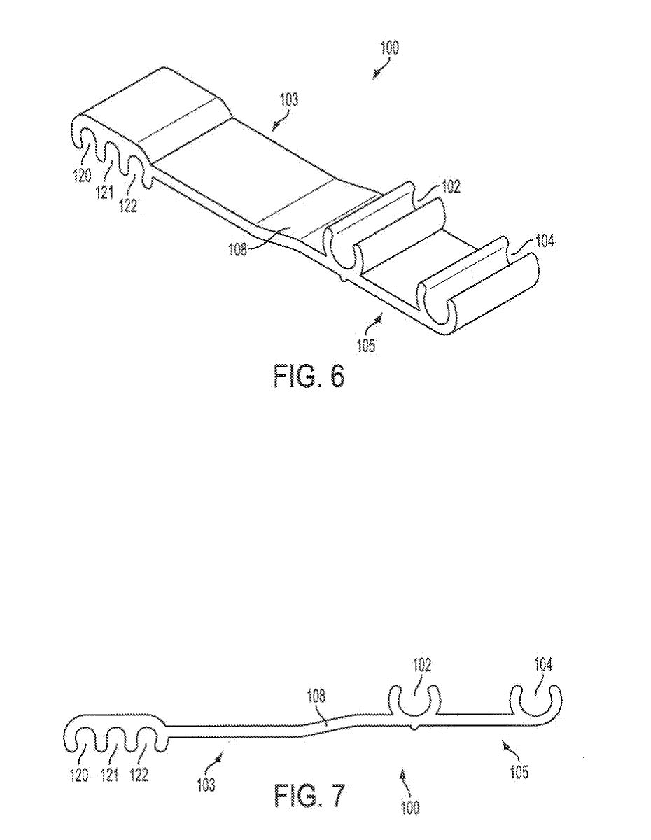

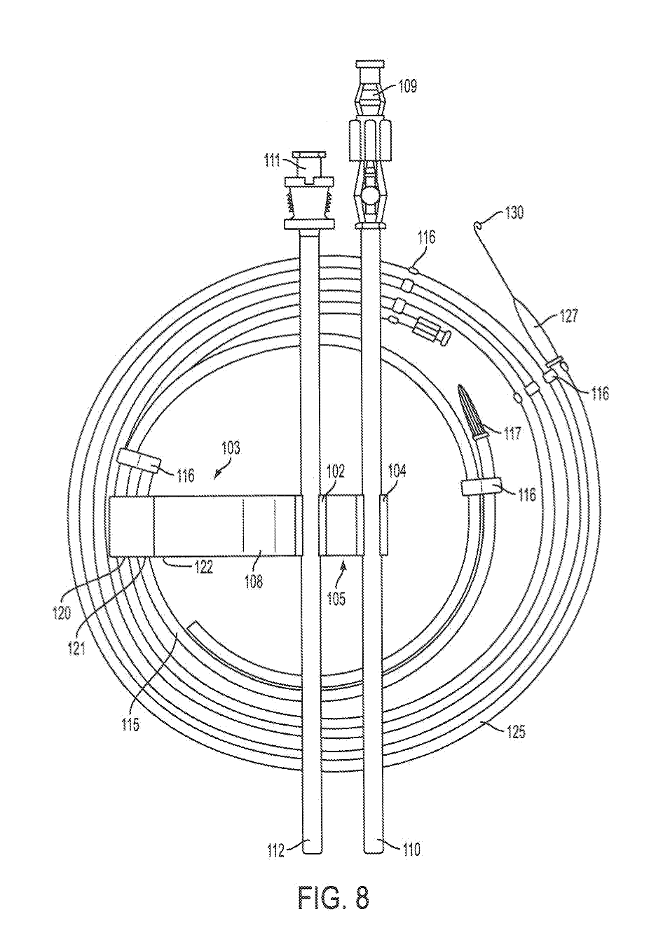

FIGS. 6 and 7 show a containment clip 100 according to a second embodiment of the invention. The containment clip 100 has a first end portion 103, a second end portion 105 and a riser portion 108 configured between the first 103 and second 105 end portions. Securement members 120, 121, 122 on the first end portion 103 face in a direction opposite of securement members 102, 104 on the second end portion 105. The containment clip 100 according to the second embodiment of the invention is modified to accommodate a non-vascular access kit, shown in FIG. 8. As explained previously above, access kits can be customized based on a number of factors, including the procedure for which the access kit is being prepared. In this case, the non-vascular access kit includes: a starter wire (not shown) contained within a starter wire hoop 115, clips 116 for retaining the form of the starter wire hoop 115, and a starter wire straightener 117; a guide wire 130 contained within a guide wire hoop 125, clips 116 for retaining the form of the guide wire hoop 125, and a guide wire straightener 127; an introducer 109 housed within an introducer protector 110; and a needle 111, housed within a needle protector 112. Compared to the containment clip 1 according to the first embodiment, the containment clip 100 according to the second embodiment has been modified to include additional securement elements 120, 121, 122 for accommodating additional kit components. A variety of modifications can be made to accommodate of kit components according to the present invention.

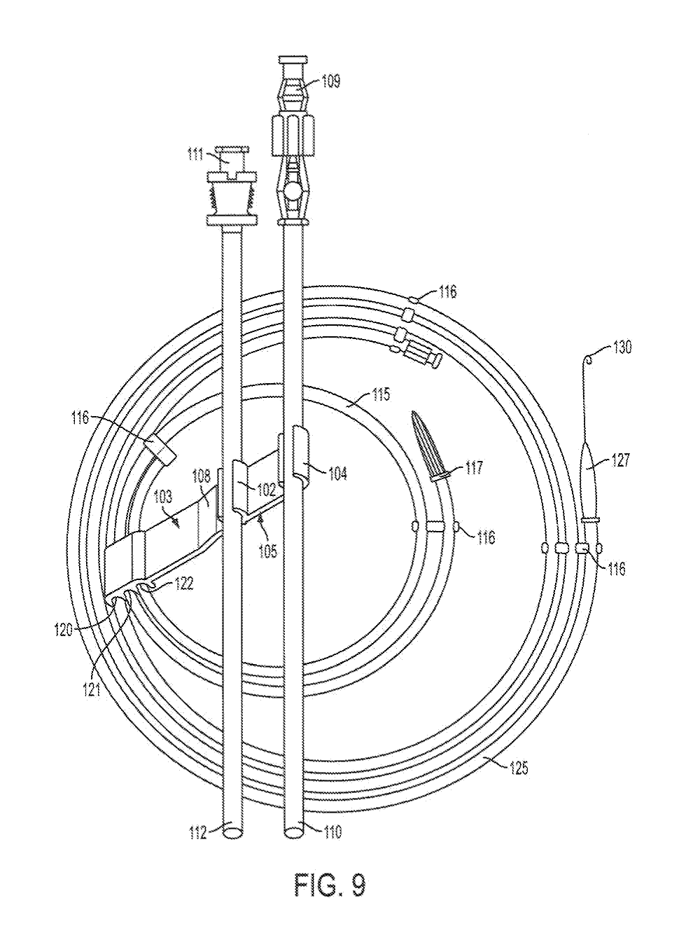

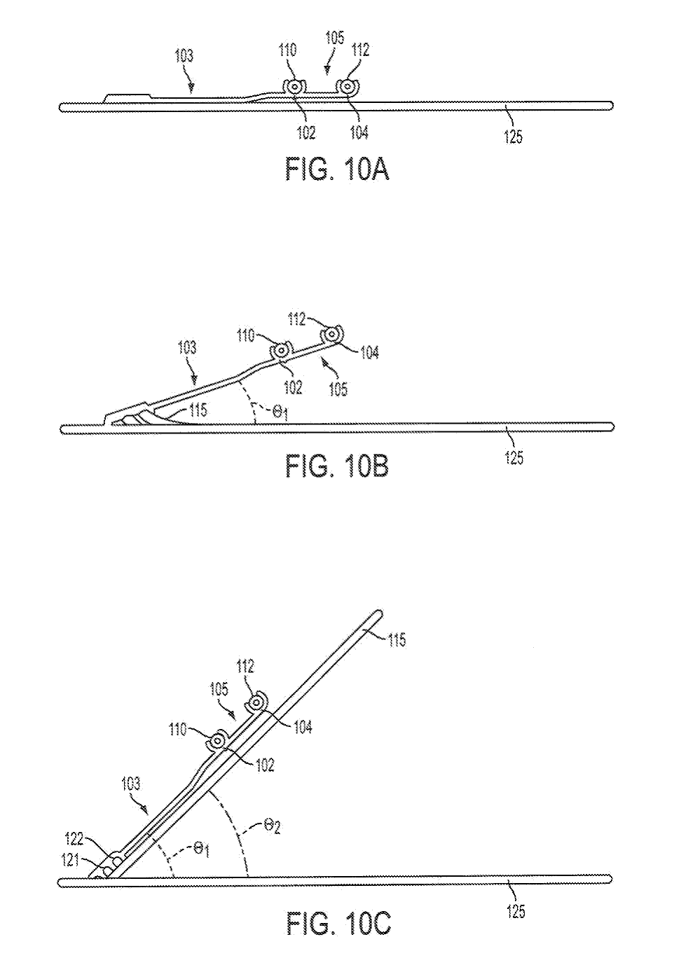

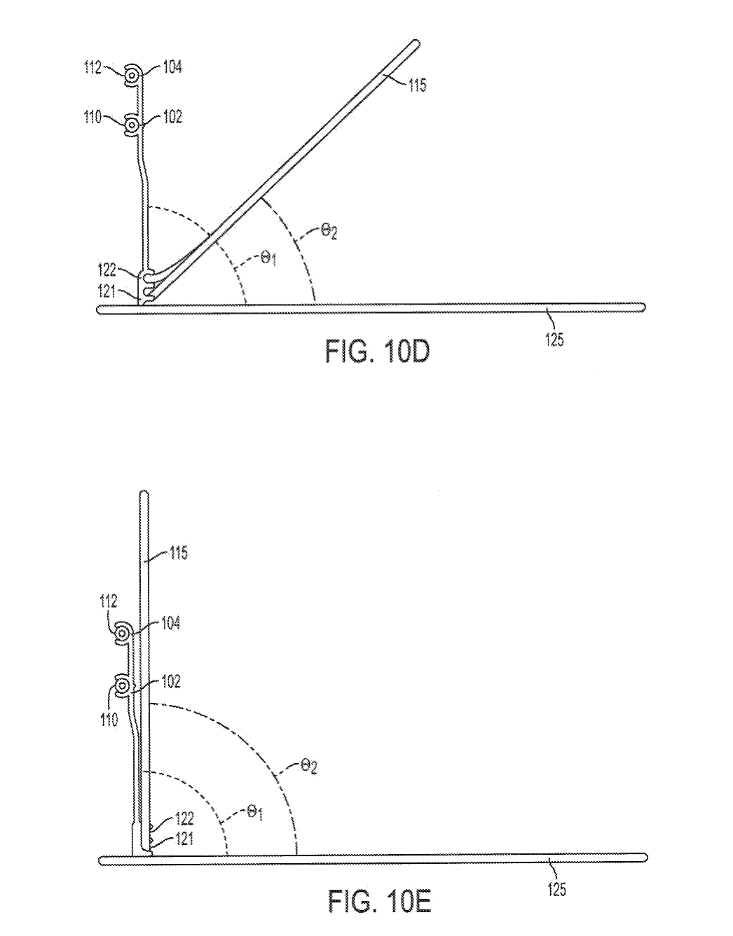

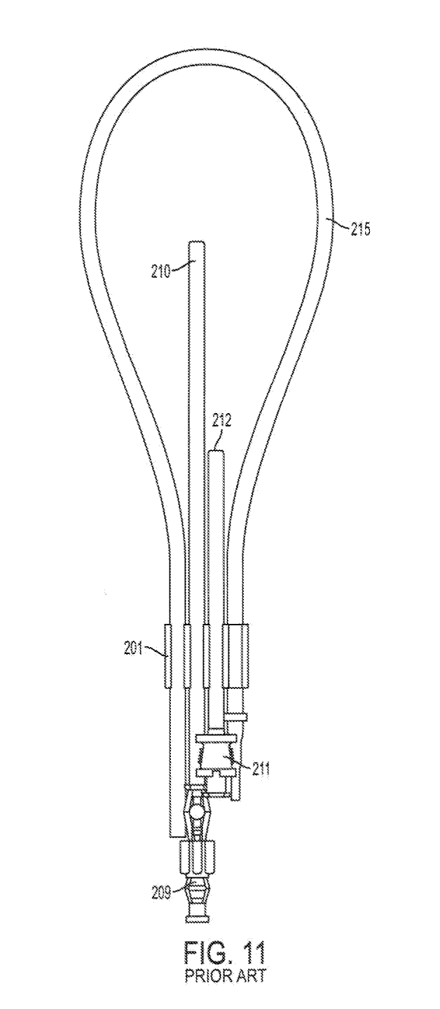

As shown in FIG. 9, the containment clip 100 forms a hinged connection with the starter wire hoop 115 and the guide wire hoop 125, and therefore the containment clip 100 can be elevated relative to the plane of the guide wire hoop 125. In one aspect, the hinged action of containment clips 100 according to this configuration can be facilitated in-part by pairing only one securement element 120, typically the outer-most securement element 120, to the outer guide wire hoop 125. This will facilitate a swinging motion and elevation for the containment clip 100. Nonetheless, since the rings of each hoop are flexible, the containment clip 100 can form a hinged connection, swing, and elevate even when multiple rings of a particular hoop are secured to the containment clip 100 (see for example FIGS. 10B and 10D, which illustrate the flexibility of the rings that make-up the hoop, permitting a hinged action for the containment clip 100).

FIGS. 10A-10E diagram various orientations of the containment clip 100 and related accessories. Similar to the containment clip 1 of the previous embodiment, the containment clip 100 of the current embodiment has securement members 120, 121, 122 that are able to stabilize the containment clip 100 in elevated positions, providing easier access to medical device accessories. For instance, the containment clip 100 can be raised by itself as shown in FIG. 10B, or it can be raised simultaneously with the starter wire hoop 115 as shown in FIG. 10C. Alternatively, for better accessory separation, the containment clip 100 can be raised past the level of the starter wire hoop 115 such that the angle Θ1 formed between the containment clip 100 and the guide wire hoop 125 is greater than the angle Θ2 formed between the starter wire hoop 115 and the guide wire hoop 125. Since the securement elements 120, 121, 122 are configured to grip the hoops with enough interference to stabilize the containment clip's 100 position, the practitioner can choose elevation levels that are comfortable and suitable for their setup. Alternatively, the practitioner can elevate both the containment clip 100 and the starter wire hoop 115 to a maximum elevation if desired.

What is claimed is:

1. A kit comprising medical device accessories, the kit comprising:
a guide wire hoop, a containment clip, and a first medical device accessory;
wherein the containment clip comprises a first and second securement member, the first securement member configured for a hinged connection to the guide wire hoop, and the second securement member configured for an interference fit with the first medical device accessory,
wherein the guide wire hoop is secured to the first securement member, and
wherein the first medical device accessory is secured to the second securement member.

2. The kit of claim 1, wherein the first and the second securement members are open-faced.

3. The kit of claim 2, wherein the first securement member and the second securement member open to opposite directions.

4. The kit of claim 2, wherein the first securement member forms a snap fit with the guide wire hoop, and the second securement member forms a snap fit with the first medical device accessory.

5. The kit of claim 1, wherein a first end portion of the containment clip comprising the first securement member and a second end portion of the containment clip comprising the second securement member are separated by a riser portion elevating the second end portion to lie in a plane different then the first end portion.

6. The kit of claim 1, wherein the second end portion comprises a second plurality of securement members.

7. The kit of claim 6, wherein a plurality of medical devices are secured to the second plurality of securement members.

8. The kit of claim 1, wherein the first end portion comprises a first plurality of securement members.

9. The kit of claim 8, wherein a plurality of medical devices are secured to the first plurality of securement members.

10. The kit of claim 9, wherein the plurality of medical devices comprises a guide wire hoop and a starter wire hoop.

11. A containment clip for securing medical device accessories, the containment clip comprising:
a first end portion, an intermediate portion, and a second end portion, the intermediate portion separating the first end portion and the second end portion;
a first open-faced securement member disposed on the first end portion; and
a second open-faced securement member disposed on the second end portion;
wherein the first securement member is configured to form a hinged interference fit with a guide wire hoop, and
wherein the second securement member is configured to form an interference fit with a medical device accessory.

12. The containment clip of claim 11, wherein the first and second securement members open to opposite directions.

13. The containment clip of claim 11, wherein the first end portion of the containment clip comprising the first securement member and the second end portion of the containment clip comprising the second securement member are separated by a riser portion elevating the second end portion to lie in a plane different than the first end portion.

14. The kit of claim 13, wherein the second end portion comprises a second plurality of securement members.

15. The kit of claim 13, wherein the first end portion comprises a first plurality of securement members.

16. The containment clip of claim 11, wherein the interference fit is a snap fit.
